(19) United States
(12) Patent Application Publication
(10) Pub. No.: US 2002/0040165 A1
(43) Pub. Date: Apr. 4, 2002

(54) Decomposition of cumene oxidation product

(76) Inventors: Richard R. Hertzog, Morristown, NJ (US); Stylianos Sifniades, Madson, NJ (US); William Bernard Fisher, Chesterfield, VA (US)

Correspondence Address: DANIEL MARK MALONEY; PEPPER HAMILTON LLP, 3000 TWO LOGAN SQUARE, 18TH AND ARCH STREETS, PHILADELPHIA, PA, 19103-2799, US

(21) Appl. No.: 09/865,190

(22) Filed: Jul. 23, 2001

Related U.S. Application Data
(63) Continuation of application No. 08/601,879, filed on Feb. 15, 1996.
Application No. 08/601,879 is a continuation of application No. 08/333,929, filed on Nov. 3, 1994, now abandoned.
Application No. 08/333,929 is a continuation of application No. 08/203,845, filed on Feb. 28, 1994, now abandoned.
Application No. 08/203,845 is a continuation of application No. 07/920,811, filed on Jul. 24, 1992, now abandoned.
Application No. 07/920,811 is a continuation of application No. 07/297,333, filed on Jan. 17, 1989, now abandoned.

(51) Int. Cl. C07C 45/42; C07C 37/08

(52) U.S. Cl. 568/383; 568/768

(57) ABSTRACT

A process for decomposing a cumene oxidation product mixture containing cumene hydroperoxide (CHP) and dimethylphenyl carbinol (DMPC) to produce phenol, acetone and alpha-methyl styrene (AMS) with enhanced safety of operation and reduced by-product formation which comprises the steps:

(a) mixing the cumene oxidation product in a stirred or back-mixed reactor with an acid catalyst, with 10 to 100 percent acetone relative to the amount of acetone produced during the decomposition reaction, and with up to 4 weight percent additional amounts of water relative to the reaction mixture, at an average temperature between about 50° C. and about 90° C. for a time sufficient to lower the average CHP concentration of the reactor to between about 0.2 and about 3.0 weight percent, and wherein a portion of DMPC is converted to dicumyl peroxide (DCP); then

(b) reacting the reaction mixture from step (a) at a temperature between about 120° C. and 150° C. under plug-flow conditions for a time sufficient to decompose substantially all residual CHP and at least 90 percent of the DCP formed in step (a).

### BACKGROUND OF THE INVENTION

1. Field of the Invention

This invention relates to a process for the preparation of phenol wherein relatively high yields of alpha-methylstyrene, a useful by-product, are obtained.

2. Description of Related Art

Phenol is manufactured via air oxidation of cumene to cumene hydroperoxide (CHP), followed by acid-catalyzed cleavage of CHP to phenol and acetone. CHP decomposition is a very exothermic reaction which is normally carried out on a commercial scale in continuous stirred or back-mixed reactors. In such reactors only a small fraction of CHP remains at any given time and the reaction medium consists essentially of the products of decomposition of CHP, i.e., phenol and acetone, plus any solvent (e.g., cumene) and other materials added with CHP to the reactor. During cumene oxidation small amounts of dimethyl phenyl carbinol (DMPC) and acetophenone are also formed. In the presence of acid catalyst, DMPC dehydrates to alpha-methylstyrene (AMS), a useful by-product. Very high yields of AMS can be obtained from pure DMPC, e.g., 98 percent yield upon dehydration over acidic silica at 300° C. In the presence of phenol, however, and more specifically in phenol/ acetone/cumene which is solvent in decomposition of technical CHP/DMPC mixtures, the AMS yield is normally about 50-60 mol percent of the DMPC. Main by-products are AMS dimers and cumylphenol which have no commercial value. Formation of cumylphenol also reduces the phenol yield.

G. G. Joris, U.S. Pat. No. 2,757,209, teaches that the amount of AMS dimers and cumylphenol formed can be substantially reduced by carrying out the reaction in two stages. In the first stage CHP is decomposed in a stirred or back-mixed reactor in the presence of small amounts of sulfur dioxide as catalyst and water as catalyst moderator. Preferred conditions are: temperature percent. Under these conditions the CHP concentration in the reaction mixture withdrawn from the reactor is less than 5 percent but more than 1 percent by weight. In the second stage, the mixture withdrawn from the first reactor is heated in a second reactor, optionally with additional catalyst, in order to decompose residual CHP and to effect the dehydration of DMPC to AMS. This second reactor is either a batch reactor, or a continuous plug-flow reactor. Preferred conditions are: temperature 110°-120° C., reaction time 5-15 minutes. Care must be taken to stop the high temperature reaction once AMS formation is completed so as to minimize dimerization of AMS or the reaction of AMS with phenol to form by-products.

U.S. Pat. No. 4,358,618, to Sifniades et al. teaches that the amount of AMS dimers and cumylphenol formed is minimized by carrying out CHP decomposition in three stages. In the first stage, CHP concentration is reduced to 0.5-5 weight percent and DMPC is converted to dicumyl peroxide (DCP) to the extent of at least 40 mol percent. The reaction is carried out in a stirred or back-mixed reactor. Preferred conditions are: temperature 50°-90° C., water 0.4-4.5 weight percent, acid 50-75 ppm. In the second stage, CHP concentration is reduced to below 0.4 weight percent by passage of the reaction mixture through a plug-flow reactor essentially isothermal to the first stage. In the third stage, both DCP and the remaining DMPC and CHP are decomposed by heating the reaction mixture to 120°-150° C. in a plug-flow reactor.

In both the aforementioned patents a key element is the presence of relatively large concentrations of residual CHP in the first stage reactor. In fact we have found that the ultimate yield of AMS from DMPC in the three stage process of U.S. Pat. No. 4,358,618 generally increases as the concentration of residual CHP in the first step is increased. Unfortunately, the higher the concentration of CHP in a stirred or back-mixed reactor, the less stable is the operation of the reactor, particularly in a large scale reactor. This is due to the fact that CHP decomposition is highly exothermic, and at the same time it is accelerated by increasing temperature. Consequently, when a relatively large concentration of residual CHP is present, the opportunity exists for a large release of thermal energy if the reaction is accelerated by a hot spot, a local surge of catalyst or other ill-controlled events. In typical commercial back-mixed reactors stable operation is very difficult to achieve at average residual CHP concentrations greater than 2-3 weight percent.

British Patent 1,202,687, to Societa' Italiana Resine S.P.A. teaches that formation of cumyl phenol and other undesirable condensates can be suppressed by carrying out CHP decomposition at 30° to 70° C. with acetone and an aqueous solution of sulfuric acid of concentration 10 - 75 weight percent resulting in a reaction product that contains 37 to 48 weight percent of acetone and 0.05 to 1.0 weight percent of sulfuric acid. The reaction is carried out in a single stage. We have found that under the broad conditions specified by said patent it is possible to obtain a reaction product that contains significant amounts of residual CHP. This is the case, for example, if the reaction is carried out at 30° C. with 10% aqueous sulfuric acid, and the resulting reaction product contains 0.05 weight percent sulfuric acid and 48 weight percent acetone. It will be appreciated, however, that mixtures containing highly reactive compounds such as CHP are not appropriate for subsequent isolation of reaction products by conventional procedures such as distillation. It is clearly the intent of said patent to effect substantially complete decomposition of CHP before product isolation. We have found that when all CHP is decomposed in a single stage within the operating conditions specified by said patent, significant amounts of cumyl phenol and AMS dimers are formed. Moreover, some DCP is also formed which in the absence of a second stage designed to decompose DCP, further decreases the yields of useful products. If operating conditions are modified towards the regime of lower reactivity (e.g. less acid, lower temperature) in an effort to suppress formation of cumyl phenol and AMS dimers, the rate of DCP formation increases. Thus the process taught in said patent cannot be used to increase the yield of useful product beyond a certain point.

### SUMMARY OF THE INVENTION

The present invention is concerned with the decomposition of cumene oxidation product in high yield to phenol, acetone and AMS, and particularly with the means of effecting such decomposition in a relatively stable and economical manner.

By adding acetone to the cumene oxidation product in a stirred or back-mixed reactor, in addition to that acetone normally produced by decomposition of CHP, relatively high yields of AMS are obtained even with residual CHP as low as 0.2 wt. percent.

The added acetone may most conveniently be obtained by adiabatic flash evaporation of crude product downstream of the process. In this way the heat content of the crude product is utilized to produce the recycle acetone and energy savings are achieved.

Therefore, the present invention includes a process for decomposing a cumene oxidation product mixture containing cumene hydroperoxide (CHP) and dimethylphenyl carbinol (DMPC) to produce phenol, acetone and alpha-methyl styrene (AMS) with enhanced safety of operation and reduced by-product formation which comprises the steps:

(a) mixing the cumene oxidation product in a stirred or back-mixed reactor with an acid catalyst, with 10 to 100 percent acetone relative to the amount of acetone produced during the reaction and with up to 4 weight percent water relative to the reaction mixture, at an average temperature between about 50° C. and about 90° C. for a time sufficient to lower the average CHP concentration of the reactor to between about 0.2 and about 3.0 weight percent and wherein a portion of DMPC is converted to dicumyl peroxide (DCP); then

(b) reacting the reaction mixture from step (a) at a temperature between about 120° and 150° C. under plug-flow conditions for a time sufficient to decompose substantially all residual CHP and at least 90% of DCP formed in step (a).

In a preferred process the product from step (b)is submitted to adiabatic flash evaporation, recovering an acetone-rich distillate which is recycled to step (a) to provide said acetone.

It may be preferred that step (a) additionally comprises reacting the reaction mixture having an average CHP concentration of between about 0.2 and about 3.0 weight percent at between 50° C. and about 90° C. under plug-flow conditions for a time sufficient to produce a reaction mixture having a CHP concentration no greater than about 0.4 weight percent.

### DESCRIPTION OF THE FIGURES

FIG. 1 shows a schematic arrangement of equipment useful in practicing the process of this invention.

FIG. 2 provides AMS yield versus CHP concentration in the stirred reactor for 60 percent, 40 percent, and no acetone recycle.

### DETAILED DESCRIPTION OF THE INVENTION

The process of the invention comprises the following steps.

(a) In a first step cumene oxidation product is mixed with an acid catalyst, with acetone and optionally with a small quantity of water in a stirred or back-mixed reactor and is held for a sufficient time to reduce the average CHP content of the reactor to between about 0.2 and about 3.0 wt percent.

(b) In a second step the effluent from step (a), is reacted at a temperature between about 120° and 150° C. for a sufficient time in a plug-flow reactor to complete the decomposition to phenol, acetone and AMS.

(c) In a third, optional step the effluent from step (b) is submitted to adiabatic flash evaporation to produce an acetone-rich distillate which is recycled to the reactor of step (a).

(d) It may be preferred that in step (a), the effluent from the back-mixed reactor is held in a plug-flow reactor for a sufficient time to reduce the CHP content to below 0.4 weight percent before proceeding with step (b).

The acid catalyst used in the process is selected from the group comprising sulfur dioxide, strong inorganic acids such as sulfuric, perchloric and the like, strong organic acids such as toluene sulfonic acid, and Lewis acids such as boron trifluoride or aluminum chloride. Typical acid catalyst levels are between 30 and 500 ppm (0.003-0.050 weight percent) of the reaction mass. The acetone used in the process may come from any convenient source. A preferred source is the adiabatic flash-evaporation of the effluent from step (b). This expedient saves the energy that would be otherwise required to evaporate the added acetone. Additionally, the flash evaporation cools the flash residue. Since the process stream must be normally cooled before neutralization of the acid catalyst, the cooling caused by flash evaporation also reduces cooling expenditures that would be otherwise required. The amount of acetone added is from about 10 percent to about 100 percent of the amount produced during the reaction. In the case of decomposition of a typical cumene oxidation product that contains 80 weight percent CHP, the recycled acetone corresponds from about 3 to about 30 weight percent of the oxidation product. Amounts less than 10 percent of that produced during the reaction have no significant beneficial effect on the reaction. Amounts higher than 100 percent are economically unattractive.

Water is normally produced during the process due to the dehydration and condensation reactions of DMPC. Additional amounts of water are introduced with the recycled acetone, especially if it is obtained by flash evaporation of the product of the plug-flow reactor. Acetone thus produced may contain from about 1 to about 5 weight percent of water. Normally the water produced in the reaction and that introduced with the acetone is sufficient to moderate the activity of the acid catalyst. If desired, small additional amounts of water may be added to further moderate the activity of the catalyst. The total amount of added water should not exceed about 4 weight percent of the reaction mixture. Too much water may cause the catalyst to become less active and slow down the reaction.

The average temperature in step (a) is from about 50° C. to about 90° C. The temperature may be maintained either by means of heat exchangers or by means of evaporative cooling. In the latter case the pressure of the reactor may be substantially below atmospheric so that the desired temperature may be achieved. The residence time is from about 5 minutes to 2 hours. It will be appreciated that the operational objective is to maintain the average CHP concentration in the reactor from between about 0.2 weight percent to about 3 wt percent. This can be achieved by an almost infinite variety of reaction conditions within the limits herein prescribed keeping in mind the following:

(a) Increasing the concentration of the acid catalyst, the temperature of the reactor and the residence time decreases the concentration of CHP.

(b) Increasing the concentration of acetone and the concentration of water increases the concentration of CHP.

If step (a) is carried out in a well-stirred reactor, both the temperature and the CHP concentration at various points of the reactor vary little from their respective average values. In such a reactor it is sufficient to monitor these quantities at only one point. If, however, a back-mixed reactor is employed, there will exist gradients of temperature and CHP concentration the magnitude of which will depend on the reactor geometry and recirculation ratio. In such a reactor the average temperature and CHP concentration are defined as the temperature and concentration that would result if the reactor contents were to be instantly homogenized. To estimate the averages it may be necessary to monitor the temperature and the CHP concentration in more than one point. Monitoring CHP is essential to the success of this process. To achieve a stable and safe operation it is preferable to have on-line analysis for CHP. This can be accomplished, for example, by means of an automatic hydroperoxide titrator, or by measuring the temperature rise in a slip-stream off the reactor circulated over a strongly acidic ion exchange resin as taught in Japanese Pat. No. 7,446,278 to Mitsui, or by any other means.

U.S. Pat. No. 4,358,618 teaches that before completing the reaction in step (b), it is desirable to ensure that the CHP content of the reaction mixture is below 0.4 weight percent. This is so, because heating CHP at the relatively high temperatures of step (b) causes a small increase in the formation of by-products. Further CHP decomposition is accomplished in step (a) by optional steps (d) which normally employs a tube with residence time of up to a few minutes. In the process of the present invention the CHP content of the effluent from step (a) may be below 0.4 weight percent. In that case step (d) is not needed. Even at high CHP levels, step (d) may be omitted as a separate piece of equipment because CHP largely decomposes during the heatup period in the heat exchanger which is part of step (b) and which may fulfill the function of step (d).

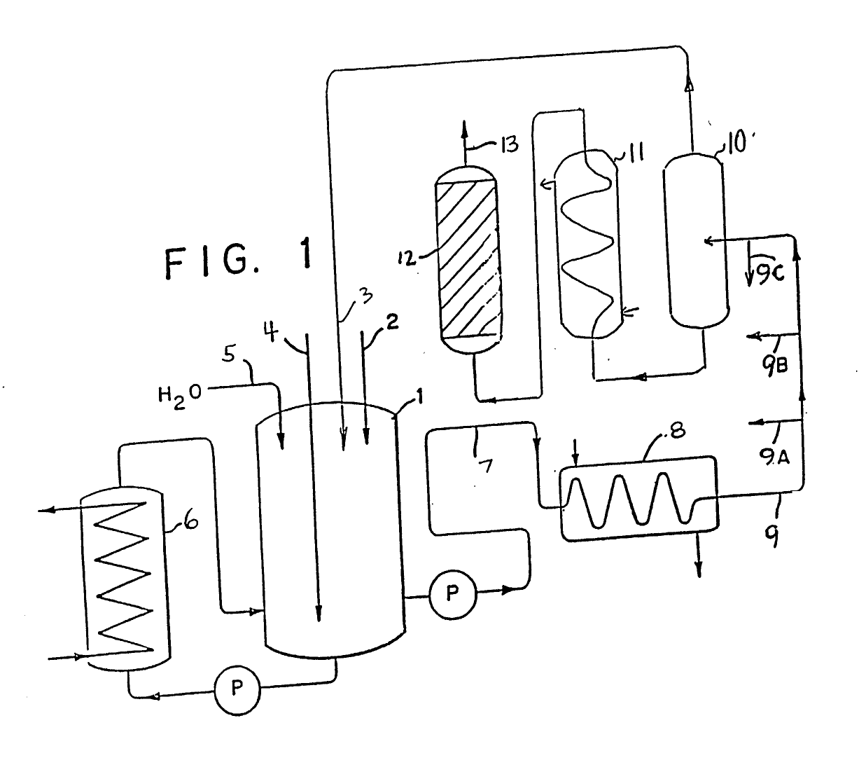

A preferred embodiment of the invention can be illustrated by reference to FIG. 1 which can be compared to FIG. 1 of U.S. Pat. No. 4,358,618. Step (a) is performed in back-mixed reactor 1 at between 50° C. and 90° C. under conditions establishing a residence time of 5-120 minutes. Technical CHP, acetone, acid catalyst and water are introduced to the reactor through inlets 2, 3, 4, 5 respectively. The desired temperature is maintained by means of circulation through cooler 6. Due to the strong exothermicity of CHP decomposition, the minimum residence time in reactor 1, which includes time spent in cooler 6, is determined by the design of the cooler and the nature of cooling fluid. A practical lower limit of approximately 5 minutes is imposed if the coolant is water. Lower residence may be achieved if a refrigeration system is employed. The upper limit of residence time depends on the temperature, the acid content, the acetone content and the water content of the reaction mixture. A residence time of approximately 120 minutes may be considered as the upper limit. Preferred residence time is between 10 and 60 minutes.

The product from reactor 1 is next pumped through tube 7 to heater 8. Optional step (d) of the reaction may carried out in tube 7 which may have sufficient capacity to provide a residence time between 0.1 and 5 minutes. The temperature in tube 7 is approximately the same as in 1. No means for cooling is provided, because the only significant reaction taking place in this step is decomposition of residual CHP which has already been reduced to 0.2 - 3 percent level in step (a). In heater 8 the product is heated to 120°-150° C. and then is pumped to flash evaporator 10 through insulated pipe 9. In heater 8 and pipe 9 step (b) is carried out, i.e. decomposition of DCP and dehydration of DMPC to AMS as well as complete decomposition of any residual CHP. The temperature at the exit of pipe 9 is normally somewhat higher than that at the exit of heat exchanger 8 due to these reactions. Best AMS yields are obtained if the residence time in heater 8 is relatively short (e.g. under 30 seconds) compared to that in pipe 9 (at least 30 additional seconds), because in that way most of the residual DMPC and DCP decompose at the higher temperature regime which favors AMS formation. Pipe 9 is fitted with sampling ports at the entrance 9A, at an intermediate point 9B and at the exit 9C, for monitoring DCP concentration. At the flash evaporator 10, step (c), the reaction product is cooled by 10°-60° C. It is further cooled to 30°-50° C. by passage through cooler 11 and then sent to an anion exchange resin bed, 12, in order to neutralize the acid catalyst. The neutralized product is then sent through pipe 13 to a distillation train for fractionation and recovery of the various components. The acetone-rich overheads from the flash evaporator are recycled to the back-mixed reactor through inlet 3.

The invention may be further illustrated by means of the following examples.

### EXAMPLE 1

A cumene oxidation product that contained 81.6 weight percent CHP, 5.00 weight percent DMPC and 0.40 weight percent acetophenone, the balance being mostly cumene, was pumped at the rate of 1.62g/min into a thermostated pyrex Morton flask equipped with magnetic stirring and an overflow device that maintained the volume of the reaction mixture at 30 mL. Simultaneously, an acetone solution containing 0.86 weight percent AMS, 2.06 weight percent phenol, 5.83 weight percent cumene, 1.60 weight percent water and 0.0512 weight percent sulfuric acid was pumped into the reactor at the rate of 0.230 g/min. The composition of the acetone solution, except for the acid content, corresponded to that of a flash distillate that could be obtained from the product of the reaction. The acetone added was equivalent to approximately 40 percent of the acetone produced during the reaction. The acid content was calculated to maintain 80 ppm in the reaction mixture. The residence time in the reactor was 16 minutes and the temperature 80° C. The stirred reactor effluent was pumped through a ⅛″ stainless steel tube reactor immersed in a bath at 125° C. The residence time in the tube was 1.6 minutes. After the two reactors had reached steady-state operation, samples were withdrawn for analysis as the exit of each reactor. Subsequently, the temperature of the bath in which the tube reactor was submerged was increased to 135° C. and again to 145° C. Samples were withdrawn for analysis in each case after the system had attained steady-state. The effluent from the stirred reactor contained on the average 2.26 weight percent DCP and 0.51 weight percent residual CHP. The yield of AMS in that effluent was 47%. The yield of AMS in the effluent of the tube reactor was 75.1, 78.0 and 78.3 mol percent respectively at 125, 135 and 145° C. The yield of AMS was computed as follows

Yield=100×(AMS-AMSO)/(CE-AMSₒ)

where AMSₒ is the concentration of AMS in the charge and CE is the total carbinol equivalent which is defined as the sum of all products that can be formed by reactions of DMPC. The main components of CE are DMPC, AMS, DCP, AMS dimers and cumylphenol.

Other examples using substantially the same cumene oxidation product but variable reaction conditions are summarized in Table I. Examples 7 through 11 utilized no added acetone and they are shown for comparison purposes. In example 12 the acetone added corresponded to 60 percent acetone recycle but the sulfuric acid was increased to 500 pm and the residence time to 20 minutes so that the residual concentration of CHP in the stirred reactor effluent was substantially zero. DCP was 0.55 weight percent. The yield of AMS in the stirred reactor effluent was 62.6% and it increased to 68.1% after further reaction at 125° C. in a tube reactor. In some examples a post-reactor isothermal to the stirred reactor was also used. This was ⅛″ stainless steel tube with residence time approximately 3 minutes.

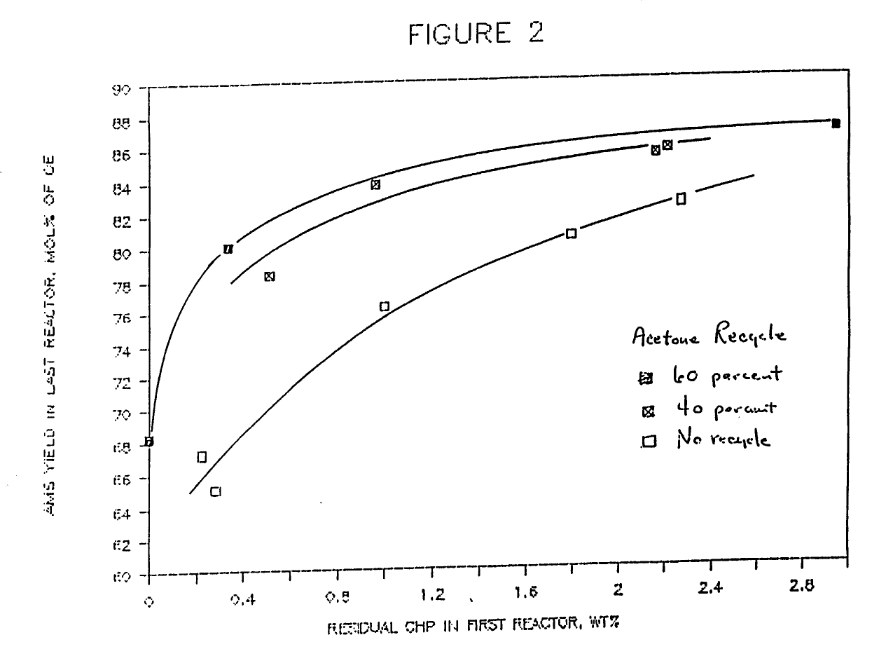

The maximum yield of AMS obtained in these examples is plotted in FIG. 2 against the concentration of residual CHP in the stirred reactor. The lowest curve (open squares) represents examples with no recycled acetone. The middle curve (squares with an x) represents examples with 40 percent acetone recycle. The upper curve (solid squares) represents examples with 60 percent acetone recycle. It is clear from the figure that by recycling acetone to the stirred reactor it is possible to obtain good AMS yields and correspondingly low residue formation at a relatively low concentration of residual CHP. It is also clear that AMS yield falls off substantially if the concentration of residual CHP is reduced below 0.2 weight percent even when acetone is recycled to the stirred reactor.

TABLE I
DECOMPOSITION OF CUMENE OXIDATION PRODUCT¹

| | | | | | | | AMS YIELD | | |
|---|---|---|---|---|---|---|---|---|---|
| | ACETONE | WATER | STIRRED REACTOR | | | POST | PLUG FLOW | | |
| | RECYCLE | ADDED | TEMP | CHP | DCP | REACTOR | TEMPERATURE, ° C. | | |
| EXAMPLE | % | % | ° C. | WT % | WT % | CHP, WT % | 125 | 135 | 145 |
| 1 | 40 | 0.21 | 80 | 0.51 | 2.26 | — | 75.1 | 78.0 | 78.3 |
| 2 | 40 | 0.21 | 70 | 0.96 | 3.75 | — | 78.3 | 82.6 | 83.7 |
| 3 | 40 | 0.90 | 80 | 2.21 | 4.71 | — | 75.7 | 84.3 | 85.7 |
| 4 | 40 | 0.90 | 80 | 2.16 | 4.27 | 0.12 | 75.7 | 84.3 | 85.4 |
| 5 | 60 | 0.08 | 80 | 0.33 | 1.77 | 0.04 | 77.3 | 79.1 | 80.1 |
| 6 | 60 | 0.90 | 80 | 2.94 | 4.32 | 0.11 | 81.0 | 86.8 | 84.1 |
| 7 | 0 | 0.03 | 70 | 0.22 | 2.73 | 0.06 | 67.2 | 66.2 | 65.0 |
| 8 | 0 | 0.03 | 70 | 0.28 | 2.60 | — | — | 65.1 | — |
| 9 | 0 | 0.84 | 70 | 1.79 | 5.80 | — | 79.8 | 80.5 | 78.4 |
| 10 | 0 | 0.84 | 80 | 1.00 | 3.61 | — | 75.7 | 76.3 | 75.1 |
| 11 | 0 | 1.34 | 80 | 2.27 | 5.17 | 0.15 | 79.3 | 82.1 | 82.4 |
| 12 | 60 | 0.10 | 80 | 0.00 | 0.55 | — | 68.1 | — | — |

¹H₂SO₄ = 80 ppm, time = 16 min except in Example 12 which had 500 ppm and 20 min respectively.

What is claimed:

1. A process for decomposing a cumene oxidation product mixture containing cumene hydroperoxide (CHP) and dimethylphenyl carbinol (DMPC) to produce phenol, acetone and alpha-methyl styrene (AMS) with enhanced safety of operation and reduced by-product formation which comprises the steps:
(a) mixing the cumene oxidation product in a stirred or back-mixed reactor with an acid catalyst, with 10 to 100 percent acetone relative to the amount of acetone produced during the decomposition reaction, and with up to 4 weight percent additional amounts of water relative to the reaction mixture, at an average temperature between about 50° C. and about 90° C. for a time sufficient to lower the average CHP concentration of the reactor to between about 0.2 and about 3.0 weight percent, and wherein a portion of DMPC is converted to dicumyl peroxide (DCP); then
(b) reacting the reaction mixture from step (a) at a temperature between about 120° C. and 150° C. under plug-flow conditions for a time sufficient to decompose substantially all residual CHP and at least 90 percent of the DCP formed in step (a).

2. The process of claim 1 wherein step (a) additionally comprises reacting the reaction mixture having an average CHP concentration of between about 0.2 and about 3.0 weight percent at between 50° C. and about 90° C. under plug-flow conditions for a time sufficient to produce a reaction mixture having a CHP concentration no greater than about 0.4 weight percent.

3. The process of claim 2 further comprising the step:
(c) submitting the reaction product from step (b) to adiabatic flash evaporation to recover an acetone-rich distillate and recycling said distillate to step (a) to provide said acetone.

4. The process of claim 3 wherein the CHP concentration is monitored by on-line analysis.

5. The process of claim 1 further comprising the step:
(c) submitting the reaction product from step (b) to adiabatic flash evaporation to recover an acetone-rich distillate and recycling said distillate to step (a) to provide said acetone.

6. The process of claim 5 wherein the CHP concentration is monitored by on-line analysis.
